(19) United States
(12) Patent Application Publication
(10) Pub. No.: US 2023/0201586 A1
(43) Pub. Date: Jun. 29, 2023

(54) COMPUTER VISION ENHANCED ELECTROMYOGRAPHY TRAINING SYSTEMS AND METHODS THEREOF

(71) Applicant: Battelle Memorial Institute, Columbus, OH (US)

(72) Inventors: David FRIEDENBERG, Columbus, OH (US); Celeste VALLEJO, Columbus, OH (US); Amanda NOONAN, Columbus, OH (US); Jordan VASKO, Columbus, OH (US)

(73) Assignee: Battelle Memorial Institute, Columbus, OH (US)

(21) Appl. No.: 18/008,337

(22) PCT Filed: Jun. 7, 2021
(86) PCT No.: PCT/US2021/036243

§ 371 Date: Dec. 5, 2022

Related U.S. Application Data
(60) Provisional application No. 63/035,276, filed on Jun. 5, 2020.
(60) Provisional application No. 63/072,619, filed on Aug. 31, 2020.

(51) Int. Cl. A61N 1/36; A61N 1/04

(52) U.S. Cl. CPC A61N 1/36031; A61N 1/0452; A61N 1/0456; A61N 1/0484

(57) ABSTRACT

EMG training systems, devices and methods are disclosed. In an approach, a computing device may receive a first input and a second input. The first input may be from an EMG device, such as the NeuroLife® sleeve provided by Battelle. A second input may be from a joint position capturing device. The computing device may create a mapping between the first input and the second input and then train a decoding algorithm based on the mapping. The decoding algorithm may be used to determine a position of the EMG device based on input received from the EMG device.

### FIELD OF INVENTION

The present application relates generally to electromyography (EMG) training systems, and more particularly to computer vision enhanced EMG training systems, devices and methods thereof.

### BACKGROUND

Limb paralysis is a common outcome of a spinal cord injury or stroke. Individuals with limb paralysis have hindered hand movement, making activities of daily living (ADLs) difficult to impossible. Neuromuscular electrical stimulation (NMES) uses electrical impulses to induce muscular contractions. NMES is defined by electrical pulses sent between electrodes and through skeletal muscles to activate a motor response. Muscle fibers in skeletal muscles respond to electrical signals sent through motor neurons. NMES induces a foreign electrical current which overrides the natural motor neuron activity and causes a muscle contraction. This may reanimate muscular movement in paralyzed limbs. NMES may also be used to enhance able limbs. Functional electrical stimulation (FES) is a subset of NMES which focuses on promoting functional movement.

Electromyography (EMG) is a diagnostic test that measures how well the muscles respond to the electrical signals emitted to specialized nerve cells called motor nerves. In electromyography (EMG) garments, electrodes are embedded in clothing to allow muscle excitation to be recorded.

A garment comprising an array of electrodes embedded therein may be configured for NMES and EMG. For example, the NeuroLife® group at the Battelle Memorial Institute has developed a high-definition NMES/EMG sleeve which both excites muscle and records muscle excitation. The NeuroLife® sleeve may allow tetraplegic individuals to regain functional hand movements. The NeuroLife® sleeve may also be used as a component in a closed-loop system for rehab for stroke, spinal cord injury, multiple sclerosis, amyotrophic lateral sclerosis, or any other injuries that disrupt normal hand/arm function.

An electromyography (EMG) system can be used to obtain EMG activities when a user is attempting to move his/her arm to which a surface EMG device, such as the NeuroLife® sleeve, is attached. A user wearing the EMG device may attempt a fixed number of gross movements (e.g. hand open, hand close). A decoding algorithm may be created and then trained to associate the EMG activities with the different movements. Currently, the trained algorithm may be able to recognize EMG activities already trained before, and thus the user can then evoke the trained movements (and only the trained movements) at will. This approach may be sufficient for individuals with motor impairments who would benefit from controlling a small number of grips, but is not be sufficient for able-bodied use cases due to the limited number of movements.

### SUMMARY

The present application discloses electromyography (EMG) training systems, devices and methods which substantially solve one or more existing technical problems due to limitations and disadvantages of the related art. In an approach, a computing device may receive a first input and a second input. The first input may be from an EMG device, such as the NeuroLife® sleeve provided by Battelle. A second input may be from a joint position capturing device. The computing device may create a mapping between the first input and the second input and then train a decoding algorithm based on the mapping. The decoding algorithm may be used to determine a position of the EMG device based on input received from the EMG device.

### BRIEF DESCRIPTION OF THE DRAWINGS

FIG. 1 is a system diagram illustrating an electromyography (EMG) training system, according to an embodiment;

FIG. 2A is an image of an EMG sleeve in an open position, according to an embodiment;

FIG. 2B is an image of the EMG sleeve of FIG. 2A as worn by a subject, according to an embodiment;

FIG. 3 is a flowchart diagram of a method for generating a decoding algorithm, according to an embodiment;

FIG. 4 is a schematic diagram of a method for decoding EMG signals, according to an embodiment;

FIG. 5A is a flowchart diagram of a method for training the decoding algorithm, according to an embodiment;

FIG. 5B is a flowchart diagram of the decoding algorithm, according to an embodiment;

FIG. 6 is a flowchart diagram of a framework for the decoding algorithm using EMG signals from multiple users, according to an embodiment;

FIG. 7 is a flowchart diagram of a development algorithm, according to an embodiment;

FIG. 8 is a flowchart diagram of a framework for the development algorithm of FIG. 7, according to an embodiment; and

FIG. 9 is a diagram illustrating predicted joint angles estimated from EMG activities, according to an embodiment.

### DETAILED DESCRIPTION OF THE PREFERRED EMBODIMENTS

EMG training systems, devices and methods that can quickly and accurately decode the full range of hand movements are disclosed. In this application, unless otherwise indicated, the terms “EMG training system” and “EMG training device” may be used interchangeably, the terms “EMG data”, “EMG signals” and “EMG activities” may be used interchangeably, and the terms “decoder” and “decoding algorithm” may be used interchangeably.

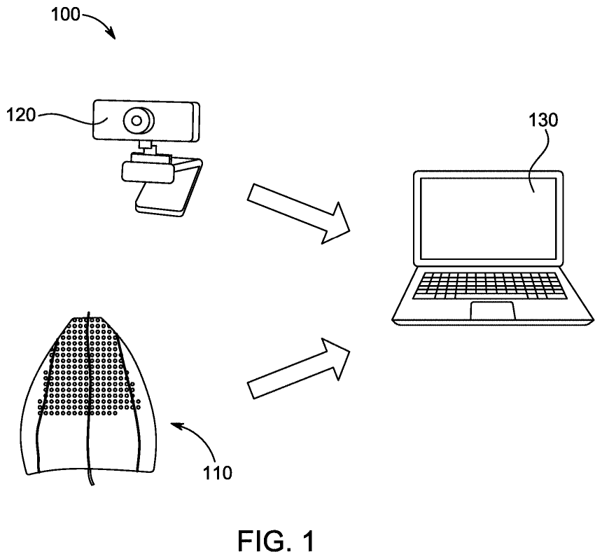

FIG. 1 is a system diagram illustrating an EMG training system 100, according to an embodiment. As shown in FIG. 1, the EMG training system 100 may comprise at least the following devices: an EMG device 100, a joint position capturing device 120, and a computing device 130.

It is noted that FIG. 1 is a schematic diagram which only illustrates the devices essential to describe this application, and other devices which are already known in this art will be omitted. The devices shown in FIG. 1 will be further described below.

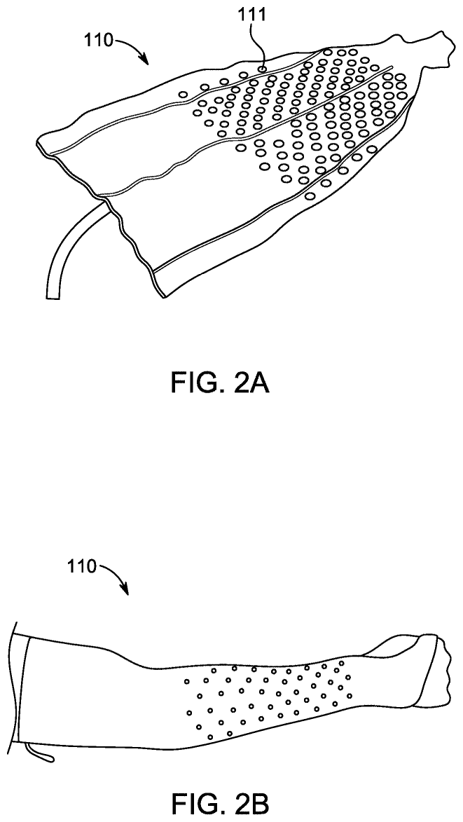

The EMG device 110 may be any device able to obtain EMG signals. As shown in FIGS. 1 and 2A-2B, in some embodiments, the EMG device 110 may be a sleeve-like device which may be used to obtain EMG signals from a user wearing this sleeve-like device. The sleeve-liked device may be any available device on the market which may be fit for the principle of this application, such as NeuroLife® sleeve provided by Battelle. The EMG device 110 may also be any EMG acquisition device currently available or developed in the future.

FIG. 2A is a sleeve-like EMG device 110 in an open position, according to an embodiment. FIG. 2B is an image of the EMG sleeve 110 as worn by a subject, according to an embodiment. The EMG sleeve 110 may comprise an array of high-definition electrodes 111 configured to contact the skin of a subject to record muscle activity. In some embodiments, the EMG sleeve 110 may be further configured to stimulate one or more muscles in the forearm. The electrodes 111 may comprise anodes and/or electrodes. In some embodiments, the electrodes 111 are relatively small to allow for fine motor recording and/or control. In some embodiments, the EMG device 110 may comprise as many as 160 electrodes 111. Each electrode 111 of the array of electrodes may comprise an anode or a cathode.

A user may freely move his/her hand/arm with the sleeve-like device across various configurations, such as hand open and hand close. If the user moves his hand/arm, then the sleeve-like device may obtain EMG data or EMG signals related to the movements of the hand/arm

It should be noted that the sleeve-like device shown in FIGS. 1, 2A and 2B is not intended to be exclusive or be limiting to the EMG device. The EMG device may be a device with any other available shape as long as it can help to realize the function discussed above and fit for the principle of this application. For example, the EMG device may be a glove-like device with the same or similar function as the sleeve-like device discussed above.

It should also be noted that the shape of the EMG device may vary based on the human body position where it is attached. For example, if a user's arm movement needs to be detected, then the sleeve-like device shown in FIGS. 1, 2A and 2B may be a good fit. If a user's finger movement needs to be detected, then a glove-like device may be desirable. If a user's leg movement needs to be detected, then a pants-like device may be used.

As shown in FIG. 1, a joint position capturing device 120 may be used to obtain position signals of the user's movements (e.g., arm movements, finger movements, etc.). The position signals may be transmitted to the computing device 130 once the joint position capturing device 120 has obtained them. The position signals may be stored in the joint position capturing device 120 and may be transmitted to the computing device 130 as needed. The position signals of the user's movements may be signals associated with joint angles, joint positions, arm angles, arm position, speed of movement, etc.

In some embodiments, the joint position capturing device 120 may be an image capturing device, such as a camera, as shown in FIG. 1. However, the camera illustrated in FIG. 1 is not intended to be exclusive or be limiting and the joint position capturing device 120 may comprise various other devices, as described in more detail below. In some embodiments, the joint position capturing device 120 may be a general-purpose camera, such as a Digital Single Lens Reflex (DSLR) camera. In some embodiments, the joint position capturing device 120 may be a camera integrated in a smart phone. In some embodiments, the joint position capturing device 120 may be a motion camera, such as a GoPro. In some embodiments, the joint position capturing device 120 may be specifically designed for capturing three-dimensional environments. The exemplary cameras provided above are not intended to be exclusive or be limiting to the present application. As will be appreciated by one having ordinary skill in the art, various other image capturing systems may be utilized.

In some embodiments, the joint position capturing device 120 may comprise a sensor device, such as the CyberGlove III provided by CyberGlove Systems or the Noitom Hi5 system provided by Noitom Ltd. These devices may provide additional information of wrist and finger joint angles, and allow collection of data in low-visibility environments. Additionally, these systems will allow tracking through complex or abnormal hand configurations, such as tracking finger movements of users with hand disabilities. These devices may be used to collect high fidelity joint angles, and may be used synergistically with computer vision techniques, such as to augment training data.

In some embodiments, the joint position tracking device 120 is an infrared (IR) device. In a further embodiment, the joint position tracking device 120 is a device comprising an IR camera. For example, the joint position tracking device 120 may comprise the Leap Motion Camera provided by Ultraleap.

The computing device 130 may be configured to process EMG signals and position signals. In some embodiments, the computing device 130 may be a computer, as illustrated in FIG. 1. The computing device 130 may process both the EMG signals from the EMG device and the position signals from the joint position capturing device 120. For example, the computing device 130 may process the position signals by using open source deep learning-based video processing algorithms, such as DeepLabCut and MediaPipe. These open source deep learning-based video processing algorithms have been proven to be useful for video tracking/video processing tasks similar to the video processing task disclosed in this application.

The computing device 130 may process the position signals (e.g., each frame of the video) from the joint position capturing device 120 and extract detailed data related to the user's hand movements (e.g., joint angles, joint positions, etc.). For example, the computing device 130 may extract joint angle measurements at wrist, elbow, fingers, etc. The more joint angle measurements extracted, the more data used for training. Therefore, this will provide a much richer training dataset for the decoding algorithm. Thus, instead of predicting from a fixed number of hand movements, the EMG training system may simultaneously predict all the joint positions, thereby capturing the full range of hand movements. In this way, the user may use the EMG device 110 to move a virtual or robotic hand using his/her EMG activities from his/her hand.

In some embodiments, the position signals are video signals. In further embodiments, video processing algorithms may be applied to process the video signals from the joint position capturing device 120 without any modification. In some embodiments, these video processing algorithms may be adapted, with some modifications, to process the video signals from the joint position capturing device 120. One benefit of using these video processing algorithms is that training data may be obtained in an efficient and cost-effective way. Further, since one of the purposes of this EMG training system is to help an able-bodied user who may freely move any part of his body (e.g., fingers, arms, etc.), accurate data may be desired because the user may be doing a small movement (e.g., a finger movement).

It will be appreciated that although computing device 130 is shown as a computer in FIG. 1, it is only given as an example of the computing device 130, and any other devices available may be used as the computing device 130 as long as it may help to realize the principle of this application. For example, the computing device 130 may be a laptop, a smart phone, etc.

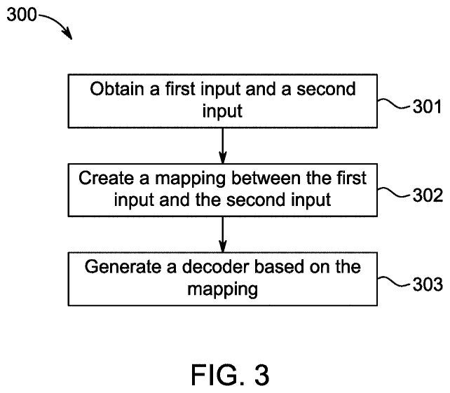

FIG. 3 is a flowchart diagram of a method for generating a decoding algorithm, according to an embodiment. In some embodiments, the method of FIG. 3 may be performed on the computing device 130 of the EMG system 100 of FIG. 1, At 301, a first input and a second input may be obtained. In an embodiment, EMG signals obtained by the EMG device 110 may be the first input and position signals obtained by the joint position capturing device 120 may be the second input. At 302, a mapping may be created between the first input and the second input. Next, a decoder may be generated based on the mapping at 303.

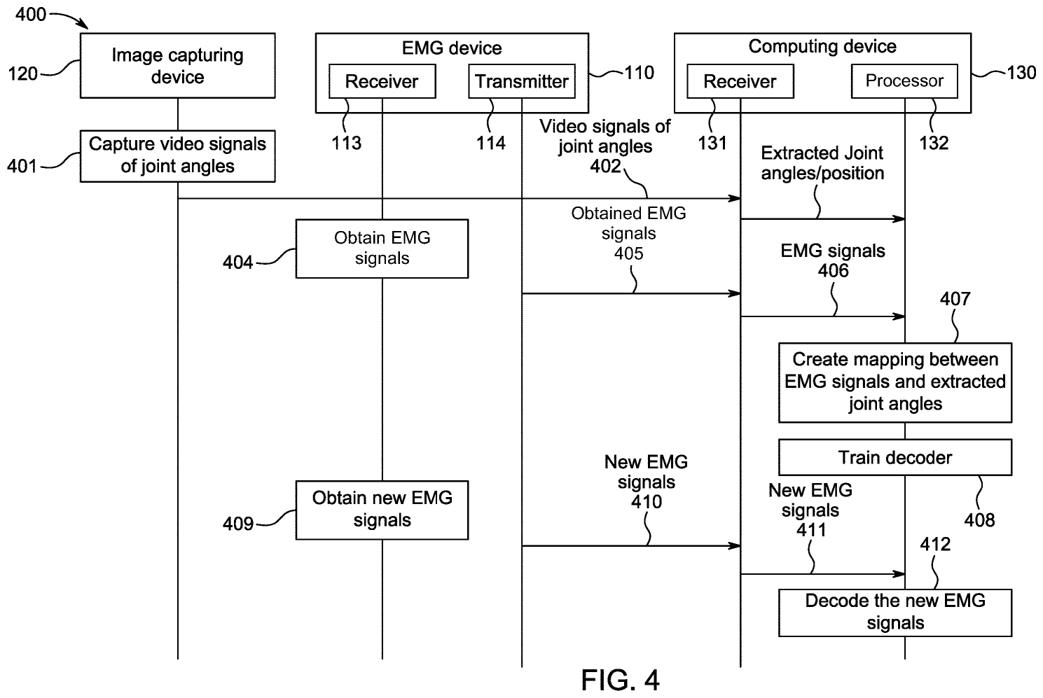

FIG. 4 is a schematic diagram of a method for decoding EMG signals of a particular user 400, according to an embodiment. The method may be executed on the EMG system 100 described above. At 401, the joint position capturing device 120 may capture position signals of joint angles. The position signals of joint angles 402 may be transmitted to a receiver 131 of the computing device 130 and 402. The receiver 131 may forward the position signals to a processor 132 of the computing device 130 at 403 to further extract joint angles and positions if necessary.

At 404, a receiver 113 of the EMG device 110 may obtain EMG signals of joint angles. At 405, a transmitter 114 of the EMG device 110 may transmit the EMG signals to the receiver 131 of the computing device 130 as a first input. Then the receiver 131 of the computing device 130 may transmit the EMG signals to a processor 132 of the computing device 130 at 406. The EMG signals may be used for training purposes described below.

In some embodiments, the EMG signals may be stored in the EMG device 110 and may be transmitted to the computing device 130 as needed. In other embodiments, the EMG signals may be transmitted to the computing device 130 once they have been obtained by the EMG device 110.

At 407, the processor 132 of the computing device 130 may create a mapping between EMG signals and joint angles extracted from the position signals. At 408, the processor 132 of the computing device 130 may then train the decoding algorithm using the mapping. Since the position signals represent physical movements (e.g., finger movements and hand movements), the mapping created by the computing device 130 may also represent a mapping between the EMG signals and a user's physical movements.

After the training, the decoding algorithm may be able to predict any known physical movement of the user by processing EMG signals from the user. For example, if the user is performing a physical movement, the receiver 113 of the EMG device 110 may obtain new EMG signals at 409 related to the physical movement. The transmitter 114 of the EMG device 110 may transmit the new EMG signals to the receiver 131 of the computing device 130 at 410, which may then forward the new EMG signals to the processor 132 at 411. At 412, the processor 132 of the computing device 130 may execute the trained decoding algorithm to predict the user's current physical movement based on the EMG signals. After recognizing the current physical movement (e.g., an arm movement) of the user, the computing device 130 may generate one or more control signals to control a virtual human body (e.g., a virtual arm) or other device which may be used to simulate the physical movement.

The trained decoding algorithm may also be used to predict the user's physical activities in real-time. For example, after the decoding algorithm recognizes a physical movement of the user, it may predict the user's next possible physical movement. The decoding algorithm may then control the virtual human body through its prediction. This movement prediction may assist the user in controlling the virtual human body in a relative easier and more efficient way.

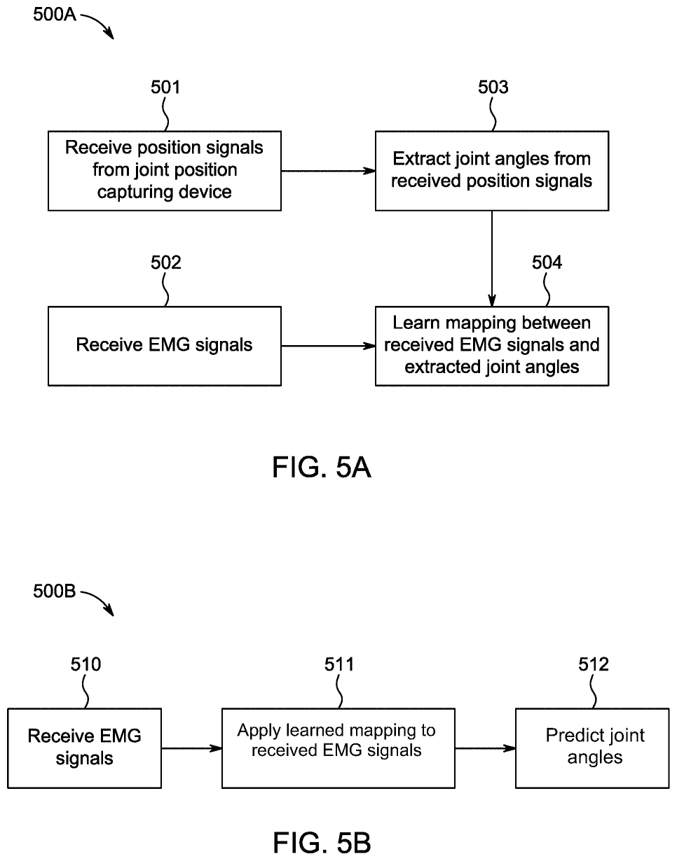

FIG. 5A is a flowchart diagram of a method for training the decoding algorithm 500A, according to an embodiment. In some embodiments, the method may be performed on the computing device 130 of the EMG system 100 of FIG. 1. At 501, position signals may be received from the joint position capturing device 120 and at 502, EMG signals may be received from the EMG device 110. At 503, joint angles may be extracted from the received position signals. At 504, a mapping between the received EMG signals and the extracted position angles may be learned.

FIG. 5B is a flowchart diagram illustrating the trained decoding algorithm 500B, according to an embodiment. In some embodiments, the decoding algorithm 500B may be executed on the computing device 130 of the EMG system 100 of FIG. 1. At 510, EMG signals may be received from the EMG device 110. At 511, the mapping learned in FIG. 5A mapping may be applied to the received EMG signals. At 512, other joint positions may be determined and/or future joint positions may be predicted. The decoding algorithm may be trained by EMG signals from multiple users. In other words, the EMG signals and position signals for the purpose of training may come from multiple users.

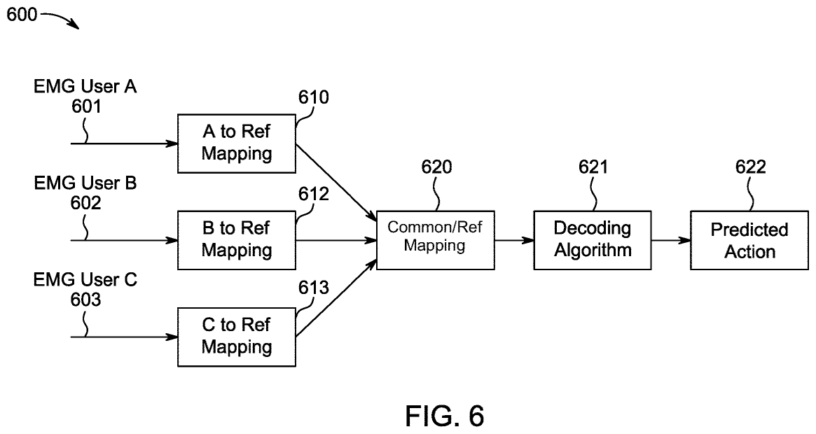

FIG. 6 is a flowchart diagram of a framework for the decoding algorithm 600 using EMG signals from multiple users, according to an embodiment. In the embodiment illustrated in FIG. 6, EMG signals are obtained from three different users, i.e., EMG user A 601, EMG user B 602, and EMG user C 603. The EMG system may learn unique transformations for each individual user, i.e., A to Reference Mapping 610 for user A 601, B to Reference Mapping 612 for user B 602, and C to Reference Mapping 613 for user C 603. The EMG system may then map the individual user data to a common “reference” user at 620. In some embodiments, there may be a plurality of reference users. At 621, the decoding algorithm trained on the reference user's data may be executed. At 622, the EMG system may then predict a future action using the trained decoding algorithm. This may reduce the amount of data required to calibrate a new user, as the decoding algorithm only needs to learn the mapping from a new user to the reference user to decode and predict a user's movements.

As shown in FIG. 6, the trained decoding algorithm may be a general-purpose decoding algorithm which may be fit for many different users, rather than a particular user. Therefore, this decoding algorithm may be applied to recognize and/or predict physical movements for different users based on principle discussed above.

In some embodiments, at least some of the multiple users used for training the decoding algorithm are able-bodied users who may freely do physical movements across various configurations. Therefore, the obtained decoder may be used to decode and/or predict an able-bodied user's movement.

In some embodiments, at least some of the multiple users used for training the decoder are persons with disability. Therefore, the obtained decoder may be used to decode and/or predict a disabled user's movement.

The decoders trained and obtained by the EMG training systems and methods may translate the EMG activities into control signals (e.g., joint angle estimates) in a fast and accurate fashion.

In one embodiment, an intermediate model (or an intermediate mapping) may be used to create mappings between EMG activities from a user (e.g., a new user) to a reference user (a prior user), thereby creating mappings between the EMG activities of different users which can then be used to decode movements of a virtual human body or other device.

For example, movements that have been learned on the reference user (e.g., a reference arm) may be projected to a user's arm to quickly learn physical activities novel to the new user. Further, movements that have been learned on the reference user may be projected to the new user's arm to quickly learn physical activities novel to the new user. This mapping could use shared-response methodology, domain adaptation, or unsupervised learning methods such as canonical correlation analysis or matrix realignment (for example, using Procrustes transformation) on the features extracted from principal component analysis and its variants, factor analysis, canonical correlation analysis or independent component analysis to learn the intermediate mapping or on unreduced EMG data. In some embodiments, the decoding algorithm may be trained for a new user using a preexisting decoding algorithm and only learning the intermediate mapping (e.g., intermediate mapping layer). In one embodiment, transfer learning may be used to quickly and efficiently build a decoder for new use cases leveraging data from old use cases. The new use cases may constitute, but are not limited to: a new user leveraging data collected from other users; a user attempting new control signals leveraging data collected from different control signals; a user at a later point in time leveraging data from an earlier point in time when EMG activity patterns may have differed; a user using an altered EMG device, such as one with damaged electrodes, leveraging data recorded before changes to the EMG device had taken place; or a user leveraging data collected for the user during training of an EMG device, such as a right arm EMG device, for training a new, related EMG device, such as a left arm EMG device. In such an algorithm, the decoder weights may be learned from a first set of data and updated using a second set of data. During the transfer learning update, some or none of the decoder weights from the initial training may be frozen such that they are not affected by the update.

In one embodiment, an image of an EMG activity may be created and then decoded by adapting a pre-trained computer vision model to this task.

In one embodiment, a self-supervised learning technique may be used to refine the trained decoders without collecting new data by learning weights on a related task that does not require data labels.

In one embodiment, a mixup or other data augmentation method may be used to boost performance from limited datasets. In such an algorithm, a first dataset may be artificially expanded to produce a second dataset that may include both the first dataset and the randomly generated examples. These new examples may be generated by an algorithm similar to mixup, for which random pairs of training examples are selected and combined in varying ratios. In the embodiment where an image of EMG activities is created, new examples may be created by rotating, flipping, cropping, shearing, or altering the color scale of the image. Data augmentation approaches are known improve decoder performance by promoting generalization to new examples and promoting robustness of the decoder to errors in the training dataset.

In some embodiments, the EMG device may be further configured to provide neuromuscular electrical stimulation (NMES). As used herein, the term “EMG/NMES device” refers to a device capable of both EMG and NMES.

NMES is defined by electrical pulses sent by electrodes and through skeletal muscles to activate a motor response. Muscle fibers in skeletal muscles respond to electrical signals sent through motor neurons. NMES induces a foreign electrical current which overrides the natural motor neuron activity and causes a muscle contraction. NMES may be used to achieve movement of paralyzed limbs. NMES may also be used to enhance movement of able limbs, for example, in sports performance enhancement and therapy. Functional electrical stimulation (FES) is a subset of NMES which focuses on promoting functional movement. In NMES devices and systems, electrodes which contact a subject's skin are activated, thereby causing a muscle contraction.

An EMG/NMES device may comprise an array of high-definition electrodes which contact the skin of a subject to stimulate one or more muscles. In some embodiments, the EMG/NMES device is a sleeve-like device, such as the NeuroLife® sleeve provided by Battelle, and may be used to stimulate muscles in the forearm. In some embodiments, a conductive medium, such as a hydrogel, may be placed between the electrode and the skin. The electrodes may comprise anodes and/or electrodes. In some embodiments, the electrodes are relatively small to allow for fine motor control. In some embodiments, the EMG/NMES device may comprise as many as 160 electrodes. Each electrode of the array of electrodes may be an anode or a cathode.

Each electrode of the array of electrodes may be configured to be inactive or active. The active electrodes are configured to generate current (i.e., be an anode) or receive current (i.e., be a cathode). As used herein, the term “pattern” refers to the specific configuration of active and inactive electrodes, as well as the amplitude and waveform of each electrode.

In some embodiments, the computing device 130 may be further configured to use the EMG signals from the EMG device and/or the position signals from the joint position capturing device 120 to train a development algorithm.

The development algorithm may determine a current skill level of a user and provide signals to improve the skill level of the user. For example, the development algorithm may provide, based on the user's determined skill level, one or more control signals which provide progressively more precise feedback and/or stimulate the muscles progressively toward more advanced movements. The feedback may work toward greater precision and less tolerance for error for the same movement. The feedback may occur in the form of signaling vibrations when the user does a movement incorrectly. Additionally, or alternatively, the feedback may stimulate the muscles to enforce the correct movement. Stimulating the muscles progressively may comprise stimulating the muscles toward more advanced movements. In some embodiments, the EMG device 110 is configured to provide NMES and the stimulation described above is achieved via NMES. In embodiments where the EMG device is configured to also provide NMES, the EMG/NMES device may utilize a specific pattern (i.e., a specific configuration of active and inactive electrodes) to provide the desired stimulation.

Additionally, or alternatively, the computing device 130 may be further configured to use the EMG signals from the EMG device and/or the position signals from the capture imaging device 120 to teach the development algorithm a new skill demonstrated by a user. The development algorithm may then teach the new skill demonstrated by a user to another user.

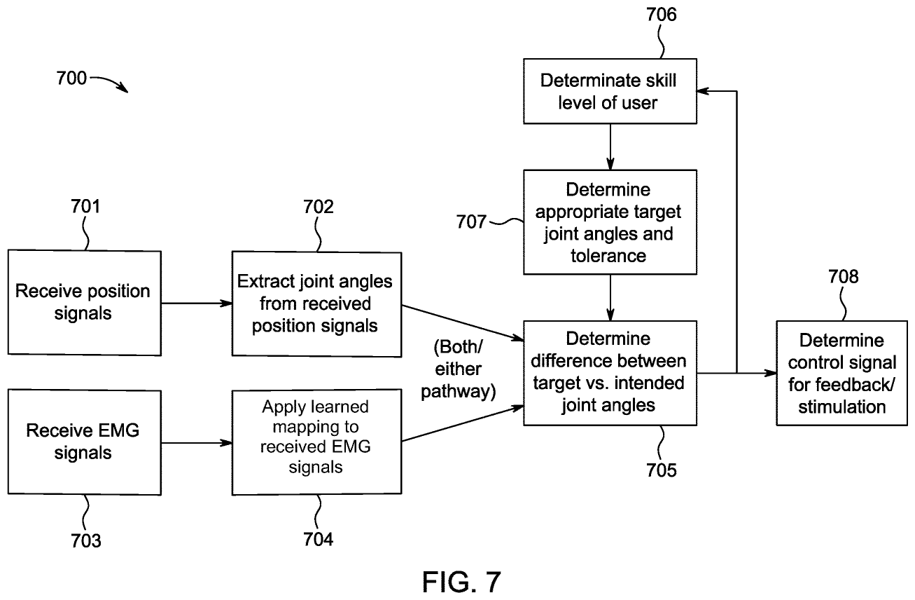

FIG. 7 is a flowchart diagram of the development algorithm 700, according to an embodiment. In some embodiments, the method may be performed on the computing device 130 of the EMG system 100 of FIG. 1. At 701 position signals are received from the joint position capturing device 130 and at 702 joint angles are extracted from the received position signals. At 703 EMG signals may be received from the EMG device 703 and at 704 a learned mapping is applied to the received EMG signals. In some embodiments, the mapping 704 may be the learned mapping 504 of FIG. 5A. In some embodiments, both 701/702 and 703/704 are executed. In other embodiments, only one of 701/702 or 703/704 is executed. At 705 the difference between the target joint angles and the intended joint angles may be determined. The difference may be used to determine a skill level of the user at 706. Using the difference and the determined skill level, appropriate target joint angles and tolerance for error may be determined at 707, which is used in 705 to determine the degree to which the intended joint angles differed from acceptable joint angles. Using the difference between the target joint angles and the intended joint angles, an appropriate control signal for feedback and/or stimulation may be determined at 708.

The development algorithm may be trained by EMG signals and/or position signals from multiple users. In other words, the EMG signals and/or position signals for the purposes of training may come from multiple users. In some embodiments, the development algorithm is trained via machine learning over time. The machine learning may comprise a series of transformations in which the EMG signals and/or position signals are compared to EMG signals and/or position signals of a reference user or multiple reference users.

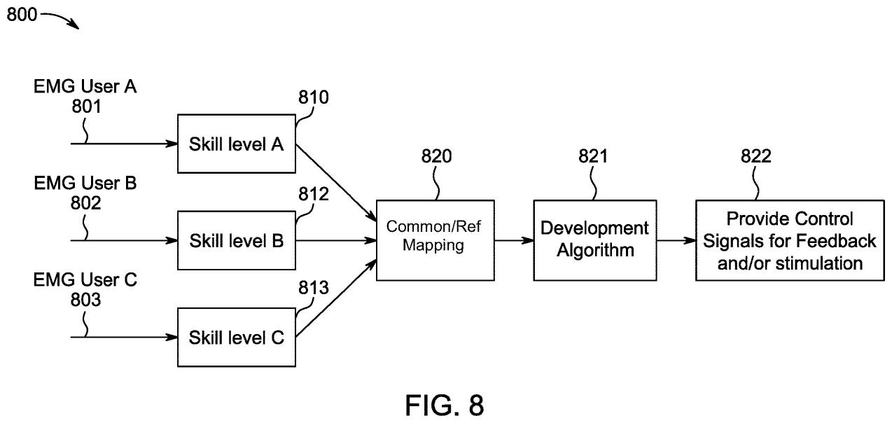

FIG. 8 is a flowchart diagram of a framework for the development algorithm 800, according to an embodiment. EMG signals may come from three different users, i.e., EMG user A 801, EMG user B 802 and EMG user C 803. The development algorithm maps the individual user data to a reference user to learn a unique skill level for each individual user, i.e., skill level A 810 for user A 801, skill level B 812 for user B 802, skill level C 813 for user C 803. At 820, the EMG system may then map the individual user data to a common “reference” user. The feedback or stimulation control signals designed to improve the skill level of the user may then be determined by the development algorithm at 821. At 822, the EMG system may then transmit one or more control signals to provide the feedback and/or stimulation.

In some embodiments, at least some of the multiple users used for training the skill algorithm have high skill movements and at least some of the multiple users used for training the skill algorithm have low skill movements. Therefore, the trained development algorithm may be utilized for a wide range of skill levels.

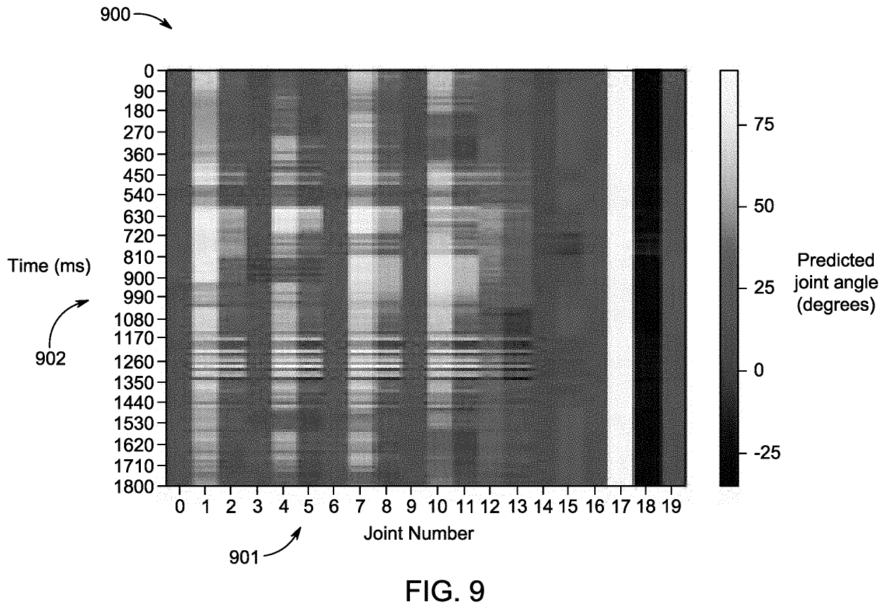

FIG. 9 is a heatmap 900 of the decoding algorithm predicting 20 continuous joint angles of a hand over the course of a 3-minute block. The x-axis 901 represents the 20 continuous joint angles and the y-axis 902 represents time in 100 ms segments. The color of each cell in the heatmap represents the predicted joint angle, in degrees from rest state. The task is a sequence of different finger and hand movements, each repeated 5x in a row. The clear alternating bands (e.g. from t=1170-1350) is indicative of the algorithm predicting joint movement, in this case hand closing and opening. It will be appreciated that this heatmap is only given by way of example. Similar heatmaps may also exist for predicting physical movements of a user.

In one embodiment, a filter, for instance a deadband filter, may be used to smooth decoder responses without slowing response times.

In one embodiment, an unsupervised or semi-supervised updating procedure may be used to continuously improve the decoding algorithm. In such decoding algorithm, decoded movements from an unsupervised recording session are used as pseudo-labels. These pseudo-labels are then used to update the decoding algorithm. Modifications to the training algorithm will be made to handle the uncertainty in prediction of the pseudo-labels.

It will be appreciated that the above-discussed hand movement embodiment with reference to FIGS. 1-9 is only given by way of example. Therefore, any available variants of the above embodiment should fall into the scope of the present application as long as those variants are consistent with the general principle of the present application. For example, the EMG training systems, devices and methods may also be used to predict leg movements of a user.

The above description discussed some embodiments regarding some improvements to the EMG training systems, devices and methods disclosed in this application. It should be appreciated that these embodiments may be varied based on the general principle of the present application. Therefore, any available variants of the above embodiments should fall into the scope of the present application as long as those variants are consistent with the general principle of the present application. It will be appreciated that the above embodiments are not necessarily independent from each other, and some of them may be incorporated within one embodiment.

Bilateral use and object manipulation will be discussed as follows. In one embodiment, the sleeve-like device shown in FIGS. 2A and 2B may be used on both arms to enable complex behaviors of the user, such as using a phone, tablet, keyboard, or manipulating objects. This data may be used to build models that predict movements and activities that people encounter during everyday use.

Therefore, based on the above description, EMG training systems, devices and methods disclosed in this application that can quickly and accurately decode the full range of hand movements may be developed.

Although features and elements are described above in particular combinations, one of ordinary skill in the art will appreciate that each feature or element can be used alone or in any combination with the other features and elements. In addition, the methods described herein may be implemented in a computer program, software, or firmware incorporated in a computer-readable medium for execution by a computer or processor. Examples of computer-readable media include electronic signals (transmitted over wired or wireless connections) and computer-readable storage media. Examples of computer-readable storage media include, but are not limited to, a read only memory (ROM), a random-access memory (RAM), a register, cache memory, semiconductor memory devices, magnetic media such as internal hard disks and removable disks, magneto-optical media, and optical media such as CD-ROM disks, and digital versatile disks (DVDs).

It will be appreciated that the terminology used in the present application is for the purpose of describing particular embodiments and is not intended to limit the application. The singular forms “a”, “the”, and “the” may be intended to comprise a plurality of elements. The terms “including” and “comprising” are intended to include a non-exclusive inclusion. Although the present application is described in detail with reference to the foregoing embodiments, it will be appreciated that those foregoing embodiments may be modified, and such modifications do not deviate from the scope of the present application.

What is claimed is:

1. A method for training an electromyography (EMG) device, the method comprising:
obtaining a first input from the EMG device and a second input from a joint position capturing device;
creating a mapping between the first input and the second input;
training a decoding algorithm based on the mapping; and
determining a position of the EMG device based on input received from the EMG device using the decoding algorithm.

2. The method of claim 1, wherein the first input is an input associated with physical movements of at least one joint of a user, an input associated with muscle activities of the user, or an input associated with EMG activity.

3. The method of claim 1, wherein the EMG device is an EMG sleeve.

4. The method of claim 1, wherein the decoding algorithm receives EMG inputs from a user, generates a mapping between the received EMG inputs and joint angles, and predicts joint angles of a user based on the mapping.

5. The method of claim 1, wherein the decoding algorithm is trained on a reference user and a new user's data is mapped to the reference user's inputs to calibrate the new user.

6. The method of claim 1, wherein training the decoding algorithm uses training data for a previous EMG device or data for the same device at one or more different points of time.

7. The method of claim 1, wherein the joint position capturing device is one of a camera or sensor device.

8. The method of claim 7, wherein the first input comprises an image of EMG activity and the decoding algorithm comprises a pre-trained computer vision model.

9. The method of claim 1, wherein the second input is an input associated with physical movements of at least one joint or an input associated with muscle activities.

10. The method of claim 1, wherein the decoding algorithm is refined by learning a mapping on a related task that does not require data labels.

11. The method of claim 1, wherein the first input, the second input, or both the first input and the second input is artificially expanded to include randomly generated examples.

12. The method of claim 1, wherein a dead band filter is used to smooth outputs of the decoding algorithm.

13. The method of claim 1, wherein decoded movements are used as pseudo-labels which are used to update the decoding algorithm.

14. The method of claim 1, further comprising determining one or more target joint angles based on a skill level of a user and determining a difference between the one or more target joint angles and one or more intended joint angles.

15. The method of claim 14, wherein further comprising determining one or more control signals for feedback or stimulation based on the determined difference.

16. The method of claim 15, wherein the stimulation is neuromuscular electrical stimulation.

17. The method of claim 15, further comprising teaching a new skill to a new user that was demonstrated by a reference.

18. An electromyography (EMG) system comprising:
an EMG device;
a joint position capturing device; and
a controller communicatively coupled to the EMG device and the joint position capturing device, the controller comprising a receiver and a processor,
the receiver configured to receive a first input from the EMG device and a second input from the joint position capturing device;
the processor configured to create a mapping between the first input and the second input;
the processor further configured to train a decoding algorithm based on the mapping;
the processor further configured to determine a position of the EMG device based on input received from the EMG device using the decoding algorithm.

19. The EMG system of claim 18, wherein the first input is an input associated with EMG activity and the second input is an input associated with physical movements of at least one joint.

20. The EMG system of claim 18, wherein the decoding algorithm receives EMG inputs from a user, generates a mapping between the received EMG inputs and joint angles, and predicts joint angles of a user based on the mapping.
